(19) United States
(12) Patent Application Publication
(10) Pub. No.: US 2010/0179386 A1
(43) Pub. Date: Jul. 15, 2010

(54) SCANNING ENDOSCOPE

(75) Inventor: Shotaro KOBAYASHI, Tokyo (JP)

Correspondence Address: GREENBLUM & BERNSTEIN, P.L.C., 1950 ROLAND CLARKE PLACE, RESTON, VA, 20191, US

(73) Assignee: HOYA CORPORATION, Tokyo (JP)

(21) Appl. No.: 12/685,109

(22) Filed: Jan. 11, 2010

(30) Foreign Application Priority Data
Jan. 13, 2009 (JP) 2009-005163

(51) Int. Cl. A61B 1/06

(52) U.S. Cl. 600/178

(57) ABSTRACT

A scanning endoscope comprising a light transmitter, a first actuator, a weight, and a second actuator, is provided. The light transmitter emits a beam of the light exiting the first emission end. The light transmitter is flexible. The first actuator is mounted near the first emission end. The first actuator bends the light transmitter in a second direction by pushing a side of the light transmitter toward the second direction. The weight is movable from the first actuator to the first emission end along the first direction. The weight changes a position of the center of mass of a protruding section by moving in unison with the light transmitter in the second direction when the first actuator bends the light transmitter. The second actuator moves the weight in either direction along the first direction.

### BACKGROUND OF THE INVENTION

1. Field of the Invention

The present invention relates to a scanning endoscope with adjustable scanning area and scanning speed.

2. Description of the Related Art

U.S. Pat. No. 6,294,775 discloses a scanning endoscope, which photographs and/or films an optical image of an observation area by scanning the observation area with light shined on a minute point in the area and successively capturing reflected light at the illuminated points.

In a general scanning endoscope, an illumination fiber that transmits light for illumination of an observation area is fixed at a certain point near an emission end of the illumination fiber. The emission end of the illumination fiber is moved by exerting a force on the illumination fiber between the emission end and the fixed point. The emission end is vibrated in two directions by adjusting the force exerted on the illumination fiber. An observation area can be scanned with light by vibrating the emission end as the emission end emits light.

In order to make the emission end vibrate in a stable manner, the illumination fiber is manipulated so that the emission end vibrates at the resonant frequency of the entire vibrating section of the illumination fiber. However, it is difficult to change the resonant frequency because the center of mass for the vibrating section of the illumination fiber is generally determined and fixed in the design process. Accordingly, it is difficult to adjust the speed of vibration even though it may be desirable to adjust the speed according to the conditions of use.

### SUMMARY OF THE INVENTION

Therefore, an object of the present invention is to provide a scanning endoscope that has an adjustable scanning speed that varies according to the speed of vibration.

According to the present invention, a scanning endoscope, comprising a light transmitter, a first actuator, a weight, and a second actuator, is provided. The light transmitter transmits light received at a first incident end to a first emission end. The light transmitter emits a beam of the light exiting the first emission end. The light transmitter is flexible. A longitudinal direction of the light transmitter is a first direction. The first actuator is mounted near the first emission end. The first actuator bends the light transmitter in a second direction by pushing a side of the light transmitter toward the second direction. The second direction is perpendicular to the first direction. The weight is movable from the first actuator to the first emission end along the first direction. The weight changes a position of the center of mass of a protruding section by moving in unison with the light transmitter in the second direction when the first actuator bends the light transmitter. The protruding section is a section of the illumination fiber that is extended from the first actuator. The second actuator moves the weight in either direction along the first direction.

According to the present invention, a scanning endoscope, comprising a light transmitter, a first actuator, and a second actuator, is provided. The light transmitter transmits light received at a first incident end to a first emission end. The light transmitter emits a beam of the light exiting the first emission end. The light transmitter is flexible. A longitudinal direction of the light transmitter is a first direction. The first actuator is mounted near the first emission end. The first actuator bends the light transmitter in a second direction by pushing a side of the light transmitter toward the second direction. The second direction is perpendicular to the first direction. The second actuator moves the light transmitter in either direction along the first direction.

### BRIEF DESCRIPTION OF THE DRAWINGS

The objects and advantages of the present invention will be better understood from the following description, with reference to the accompanying drawings in which:

FIG. 1 is a schematic diagram of a scanning endoscope apparatus comprising a scanning endoscope of the first embodiment of the present invention;

FIG. 2 is a block diagram schematically showing the internal structure of the scanning endoscope processor;

FIG. 3 is a block diagram schematically showing the internal structure of the scanning endoscope;

FIG. 4 is a cutaway view along the axial direction of the hollow tube schematically showing the structure of the fiber driving unit of the first embodiment;

FIG. 5 is a cutaway view of the hollow tube along the axial direction of the hollow tube schematically showing the support structure for the illumination fiber in the first embodiment;

FIG. 6 is a graph illustrating the changing position of the emission end in the second and third directions;

FIG. 7 is an illustration of a spiral course along which the emission end of the illumination fiber is moved by the actuator;

FIG. 8 is a schematic side-view of the actuator and the illumination fiber showing the movement of the illumination fiber toward the distal end of the insertion tube in the first embodiment;

FIG. 9 is an illustration of the change in the scanned area according to the distance between the emission end and the observation area;

FIG. 10 is a schematic side-view of the actuator and the illumination fiber showing the movement of the illumination fiber away from the distal end of the insertion tube in the first embodiment;

FIG. 11 is an illustration of light being emitted from the lens and striking the observation area;

FIG. 12 is a cutaway view along the axial direction of the hollow tube schematically showing the structure of the fiber driving unit of the second embodiment;

FIG. 13 is a schematic side-view of the actuator and the illumination fiber showing the movement of the fiber supporter toward the distal end of the insertion tube in the second embodiment;

FIG. 14 is a schematic side-view of the actuator and the illumination fiber showing the movement of the fiber supporter away from the distal end of the insertion tube in the second embodiment;

FIG. 15 is a cutaway view along the axial direction of the hollow tube schematically showing the structure of the fiber driving unit of the third embodiment;

FIG. 16 is a schematic side-view of the actuator and the illumination fiber showing the movement of the illumination fiber and the fiber supporter toward the distal end of the insertion tube in the third embodiment;

FIG. 17 is a schematic side-view of the actuator and the illumination fiber showing the movement of the illumination fiber and the fiber supporter away from the distal end of the insertion tube in the third embodiment; and

FIG. 18 is a cutaway view along the axial direction of the hollow tube schematically showing the structure of the fiber driving unit of the fourth embodiment.

### DESCRIPTION OF THE PREFERRED EMBODIMENTS

The present invention is described below with reference to the embodiment shown in the drawings.

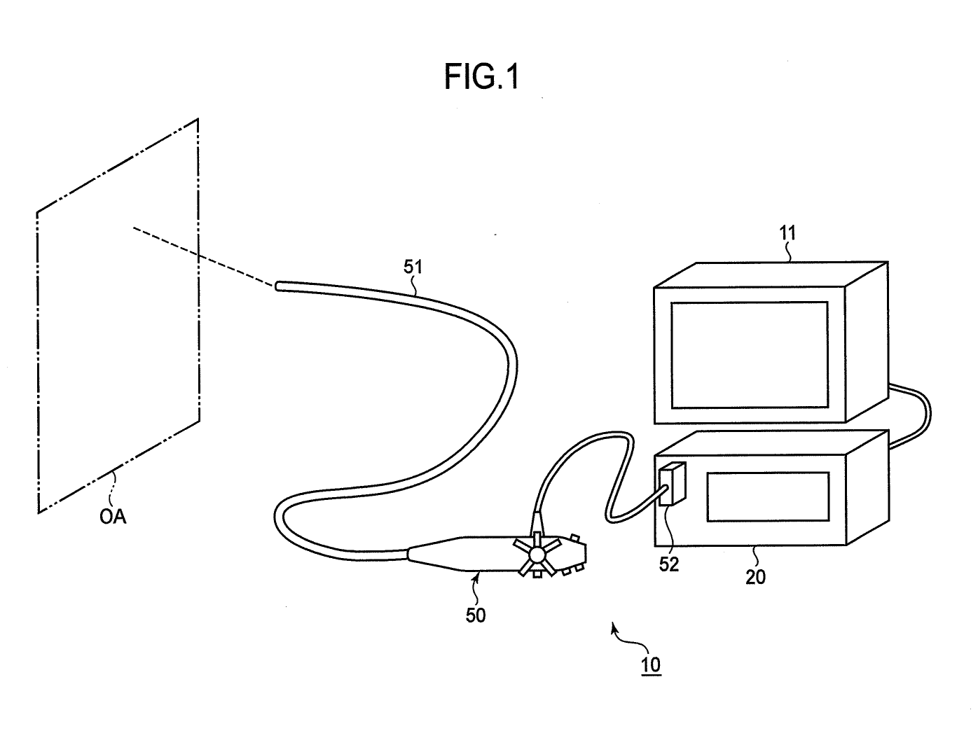

In FIG. 1, the scanning endoscope apparatus 10 comprises a scanning endoscope processor 20, a scanning endoscope 50, and a monitor 11. The scanning endoscope processor 20 is connected to the scanning endoscope 50 and the monitor 11.

Hereinafter, an emission end of an illumination fiber (not depicted in FIG. 1) and incident ends of image fibers (not depicted in FIG. 1) are ends mounted in the distal end of the insertion tube 51 of the scanning endoscope 50. In addition, an incident end of the illumination fiber (first incident end) and emission ends of the image fibers are ends mounted in a connector 52 that connects to the scanning endoscope processor 20.

The scanning endoscope processor 20 provides light that is shined on an observation area (see “OA” in FIG. 1). The light emitted from the scanning endoscope processor 20 is transmitted to the distal end of the insertion tube 51 through the illumination fiber (light transmitter), and is directed towards one point in the observation area. Light reflected from the illuminated point is transmitted from the distal end of the insertion tube 51 to the scanning endoscope processor 20.

The direction of the emission end of the illumination fiber (first emission end) is changed by an actuator (not depicted in FIG. 1). By changing the direction, the observation area is scanned with the light emitted from the illumination fiber. The actuator (first actuator) is controlled by the scanning endoscope processor 20.

The scanning endoscope processor 20 receives reflected light that is scattered at the illuminated point, and generates a pixel signal according to the amount of received light. One frame of an image signal is generated by generating pixel signals corresponding to the illuminated points dispersed throughout the observation area. The generated image signal is transmitted to the monitor 11, where an image corresponding to the received image signal is displayed.

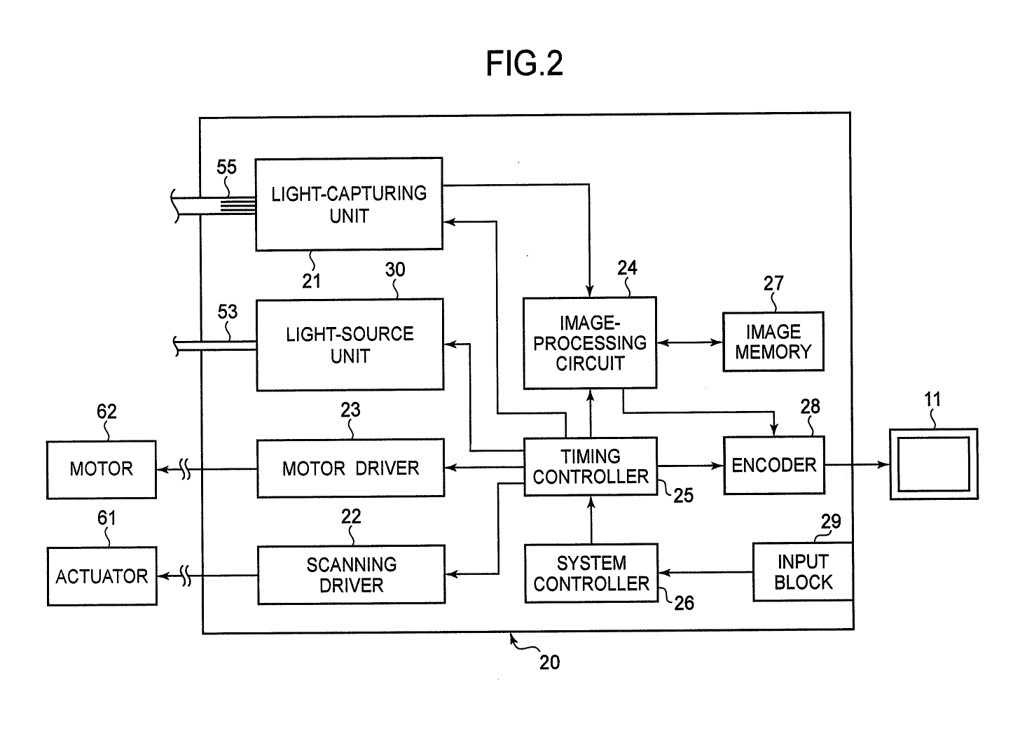

As shown in FIG. 2, the scanning endoscope processor 20 comprises a light-source unit 30, a light-capturing unit 21, a scanning driver 22, a motor driver 23, an image-processing circuit 24, a timing controller 25, a system controller 26, and other components.

The light-source unit 30 comprises red, green, and blue lasers (not depicted) that emit red, green, and blue laser beams, respectively. The red, green, and blue laser beams are mixed into white light, which is emitted from the light-source unit 30.

The white light emitted from the light-source unit 30 is supplied to the illumination fiber 53. The scanning driver 22 controls the actuator 61 so that the emission end of the illumination fiber 53 traces a predetermined course. The motor driver 23 controls a motor 62 (second actuator, third actuator) to adjust a length of the illumination fiber that extends from the actuator 61, as described later.

The reflected light at the illuminated point within the observation area is transmitted to the scanning endoscope processor 20 by the image fibers 55 mounted in the scanning endoscope 50. The transmitted light is made incident on the light-capturing unit 21.

The light-capturing unit 21 generates a pixel signal according to the amount of the transmitted light. The pixel signal is transmitted to the image-processing circuit 24, which stores the received pixel signal in the image memory 27. Once pixel signals corresponding to the illuminated points dispersed throughout the observation area have been stored, the image-processing circuit 24 carries out predetermined image processing on the pixel signals, and then one frame of the image signal is transmitted to the monitor 11 via the encoder 28.

By connecting the scanning endoscope 50 to the scanning endoscope processor 20, optical connections are made between the light-source unit 30 and the illumination fiber 53 mounted in the scanning endoscope 50, and between the light-capturing unit 21 and the image fibers 55.

In addition, by connecting the scanning endoscope to the scanning endoscope processor 20, electrical connections are made between the actuator 61 mounted in the scanning endoscope 50 and the scanning driver 22, and between the motor 62 mounted in the scanning endoscope and the motor driver 23.

The timing for carrying out the operations of the light-source unit 30, the light-capturing unit 21, the scanning driver 22, the motor driver 23, the image-processing circuit 24, and the encoder 28 is controlled by the timing controller 25. In addition, the timing controller 25 and other components of the endoscope apparatus 10 are controlled by the system controller 26. A user can input some commands to the input block 29, which comprises a front panel (not depicted) and other mechanisms.

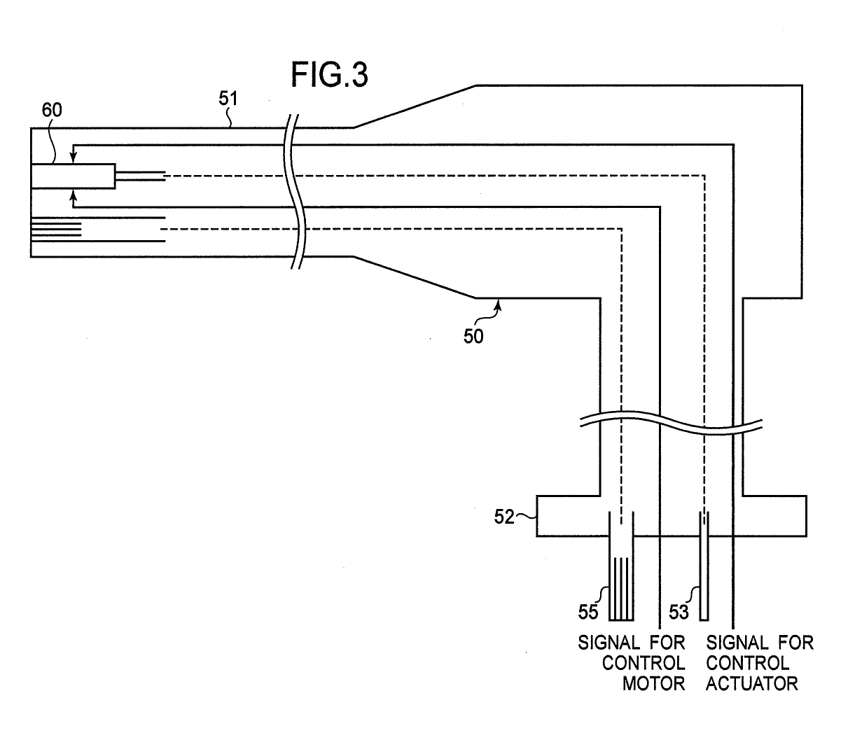

Next, the structure of the scanning endoscope 50 is explained. As shown in FIG. 3, the scanning endoscope 50 comprises the illumination fiber 53, a fiber driving unit 60, the image fibers 55 and other components.

The illumination fiber 53 and the image fibers 55 are arranged inside the scanning endoscope 50 from the connector 52 to the distal end of the insertion tube 51.

As described above, a laser beam of the white light emitted by the light-source unit 30 is made incident on the incident end of the illumination fiber 53. The incident white light is then transmitted to the emission end of the illumination fiber 53.

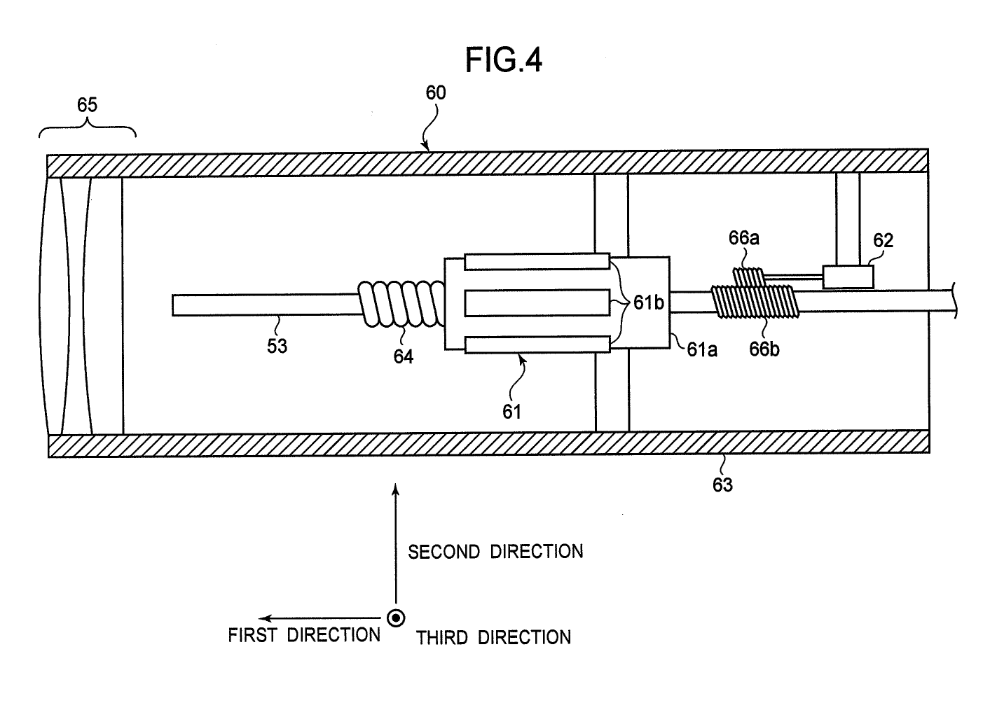

The fiber driving unit 60 is mounted at the distal end of the insertion tube 51. As shown in FIG. 4, the fiber driving unit 60 comprises the actuator 61, the motor 62, a rigid tube 63, a fiber supporter 64 (weight), and other components.

The rigid tube 63 is made from rigid material. The rigid tube 63 is mounted at the distal end of the insertion tube 51. The rigid tube 63 is positioned so that the axial direction of the tube is parallel to a first direction that is an axial direction of the insertion tube 51 at the distal end. A lens 65 is mounted at the end of the rigid tube 63 nearest to the distal end of the insertion tube 51.

The actuator 61 comprises a hollow tube 61a and piezoelectric elements 61b. The hollow tube 61a is made from flexible material. The piezoelectric elements 61b are mounted on an outside surface of the hollow tube 61a. The flexible hollow tube 61a is formed so that the outside diameter of the hollow tube 61a is less than the inside diameter of the rigid tube 63.

The hollow tube 61a is fixed inside the rigid tube 63 so that the axes of both the hollow tube 61a and the rigid tube 63 coincide with each other. A section of the hollow tube 61a near the end opposite to the distal end of the insertion tube 51 is fixed to the rigid tube 63.

Four piezoelectric elements 61b are adhered to the hollow tube 61a so that all piezoelectric elements 61b can expand and contract along the first direction. A pair of piezoelectric elements is arranged along a second direction perpendicular to the first direction so that the axis of the hollow tube 61a is between the pair of piezoelectric elements. In addition, the other two piezoelectric elements 61b are arranged along a third direction perpendicular to the first and second direction so that the axis of the hollow tube 61a is between the piezoelectric elements.

The piezoelectric elements 61b expand and contract along the first direction on the basis of a scan control signal transmitted from the scanning driver 22 to the piezoelectric elements 61b. The hollow tube 61b bends in a direction, which is perpendicular to the first direction, by adjustments in the expansion and contraction pattern of the four piezoelectric elements 61b.

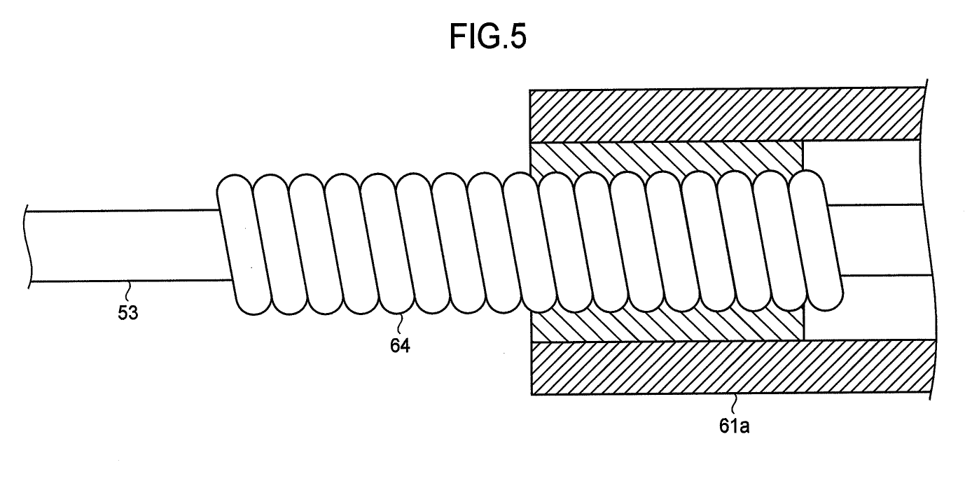

The fiber supporter 64 is made of metal and configured as a coil spring with an inside diameter of the coil that is practically equal to the outside diameter of the illumination fiber 53. As shown in FIG. 5, the fiber supporter 64 is partially housed and secured inside of the hollow tube 61a so that the axis of the coil coincides with the axis of the hollow tube 61a, and so that a section of the fiber supporter 64 protrudes from the hollow tube toward the distal end of the insertion tube 51.

The illumination fiber 53 passes through the coil-shaped fiber supporter 64. The illumination fiber 53 is supported by the fiber supporter 64 as the emission end of the illumination fiber 53 protrudes from the fiber supporter 64. Accordingly, the fiber supporter 64 is positioned between the actuator 61 and the illumination fiber 53. The illumination fiber 53 is not fixed to the fiber supporter 64 and is free to move in the first direction.

When the actuator 61 is deflected to one side, the fiber supporter 64 is deformed elastically before the fiber supporter 64 starts restoring its original form. While in the process of restoring its original form, a pushing force is transmitted by the actuator 61 to the side of the illumination fiber 53.

The illumination fiber 53 is flexible. The side of illumination fiber 53 is pushed along the second and/or third directions by the actuator 61 via the fiber supporter 64, and the illumination fiber 53 bends toward the second and/or third directions, which are perpendicular to the longitudinal direction of the illumination fiber 53. The emission end of the illumination fiber 53 is moved by bending the illumination fiber 53.

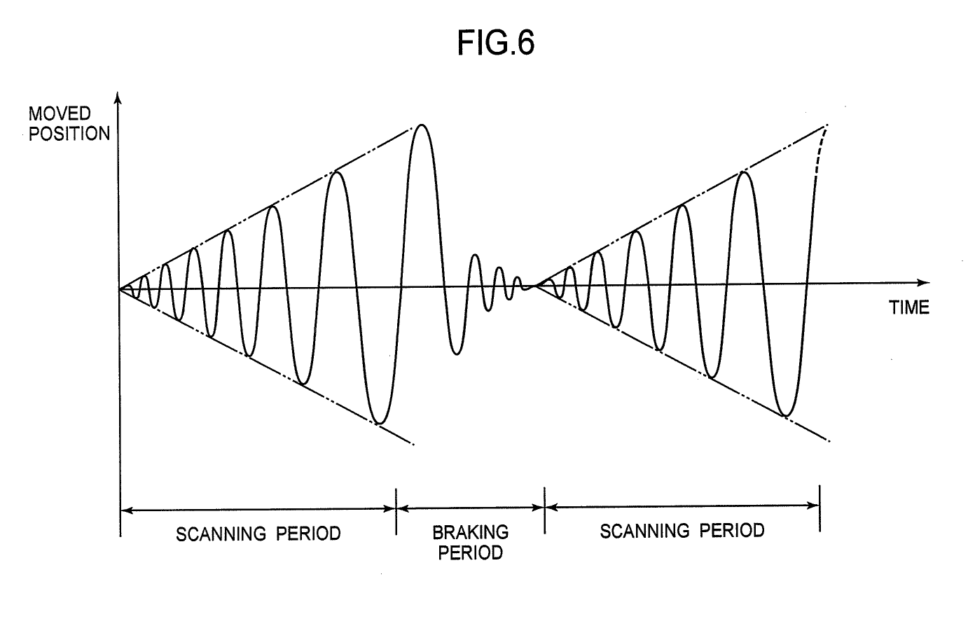

As shown in FIG. 6, the emission end of the illumination fiber 53 is moved so that the emission end vibrates along the second and third directions at amplitudes that are repetitively increased and decreased. The frequencies of the vibration along the second and third directions are adjusted to be equal. In addition, the periods of increasing and decreasing amplitudes of vibration in the second and third directions are synchronized. Further, the phases of the vibration in the second and third directions are shifted by 90 degrees.

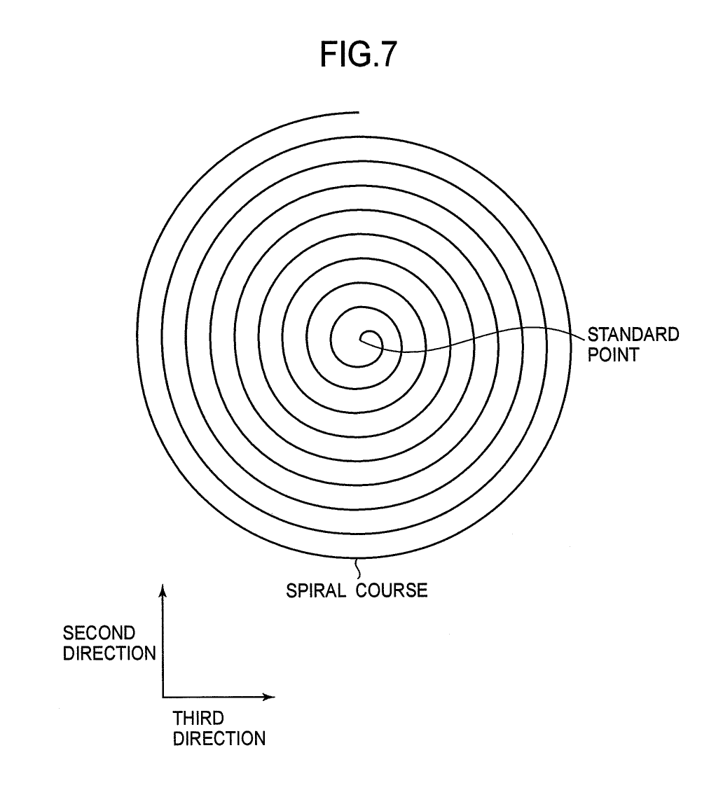

By vibrating the emission end of the illumination fiber 53 along the second and third directions as described above, the emission end traces the spiral course shown in FIG. 7, and the observation area is scanned with the white laser beam.

As shown in FIG. 4, the motor 62 is fixed in the rigid tube 63 at a position further from the distal end of the insertion tube 51 than the actuator 61. The motor 62 is connected to a first worm gear 66a. The motor 62 rotates the first worm gear 66a on a straight line parallel to the first direction.

A second worm gear 66b is wound around the illumination fiber 53 at a position nearer to the incident end of the illumination fiber 53 than the fiber supporter 64. The first and second worm gears 66a and 66b are figured and positioned so that the first and second worm gears 66a and 66b mesh together.

The second worm gear 66b is rotated when the motor 62 rotates the first worm gear 66a. By rotating the second worm gear 66b, the illumination fiber 53 within the second worm gear 66b moves either toward or away from the distal end of the insertion tube 51 along the first direction.

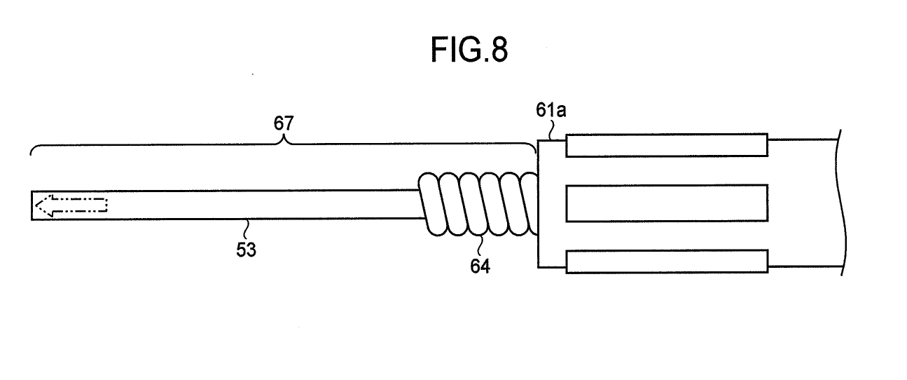

The protruding section 67, which extends from the hollow tube 61a, and the fiber supporter 64 vibrate together as one body. When the illumination fiber 53 moves toward the distal end of the insertion tube 51 (see FIG. 8), the center of mass of the protruding section 67 shifts toward the distal end of the insertion tube 51. In addition, the resonant frequency of the protruding section 67 is decreased by extending the length of the section of the illumination fiber 53 that is vibrated. The scanning driver 22 controls the actuator 61 so that the frequency of the vibration of the illumination fiber 53 coincides with the resonant frequency of the protruding section 67. By decreasing the frequency of vibration, the scanning speed can be reduced.

In addition, when the illumination fiber 53 moves toward the distal end of the insertion tube 51, the emission end of the illumination fiber 53 approaches the lens 65. By moving the emission end toward the lens 65, the distance between the emission end and the observation area is reduced.

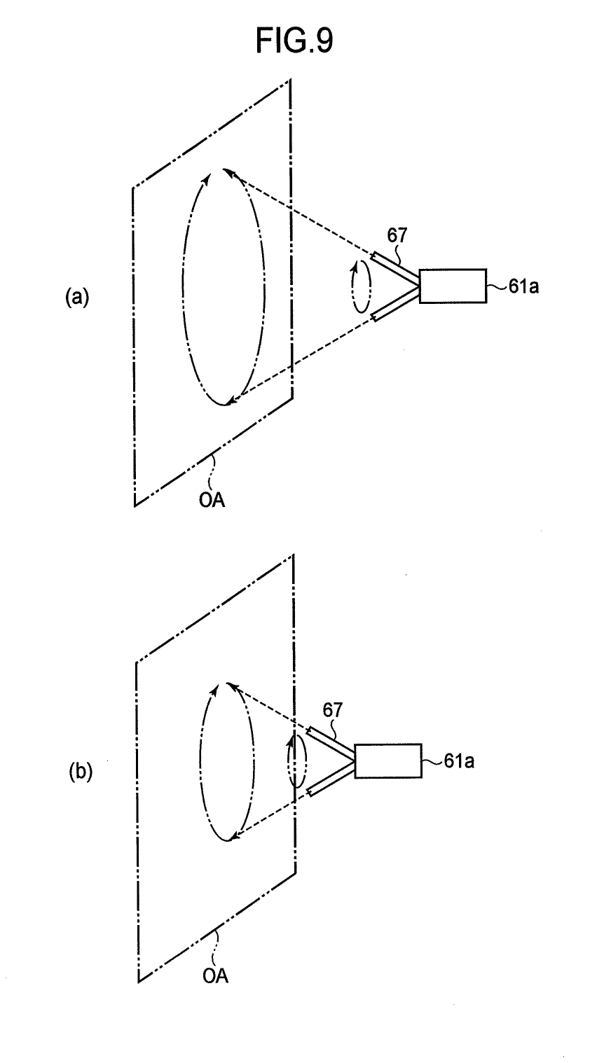

As shown in FIG. 9(a), when the distance between the emission end and the observation area (see “OA”) is longer, a scanned area is enlarged. On the other hand, as shown in FIG. 9(b), when the distance between the emission end and the observation area is shorter, a scanned area becomes smaller.

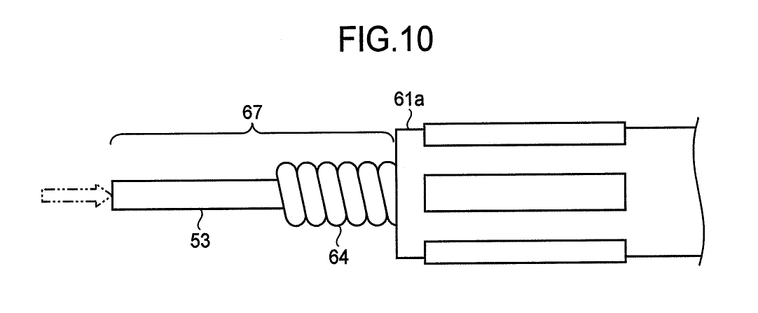

When the illumination fiber 53 moves away (recedes) from the distal end of the insertion tube 51 (see FIG. 10), the center of mass of the protruding section 67 is shifted along the first direction away from the distal end of the insertion tube 51. In addition, the length of the section of the illumination fiber 53 that vibrates is shortened. Accordingly, the resonant frequency of the protruding section 67 increases. The scanning driver 22 controls the actuator 61 so that the frequency of the vibration of the illumination fiber 53 coincides with the resonant frequency of the protruding section 67. By increasing the frequency of vibration, the scanning speed can increase.

In addition, when the illumination fiber 53 moves away from the distal end of the insertion tube 51, the emission end of the illumination fiber 53 moves away from the lens 65. By moving the emission end away from the lens 65, the distance between the emission end and the observation area increases. Accordingly, a scanned area is enlarged.

The position of the emission end of the illumination fiber 53 when the illumination fiber 53 is not deflected is defined as a standard point (see FIG. 7). While the emission end is vibrated with increasing amplitude starting from the standard point (see “scanning period” in FIG. 6), illumination of the observation area with the white laser beam and generation of pixel signals are carried out.

In addition, when the amplitude reaches a maximum among the predetermined range, one scanning operation for producing one image terminates. After termination of a scanning operation, the emission end of the illumination fiber 53 is returned to the standard point by vibrating the emission end with decreasing amplitudes (see “braking period” in FIG. 6). When the emission end is returned to the standard point, it is the beginning of a scanning operation for generating another image.

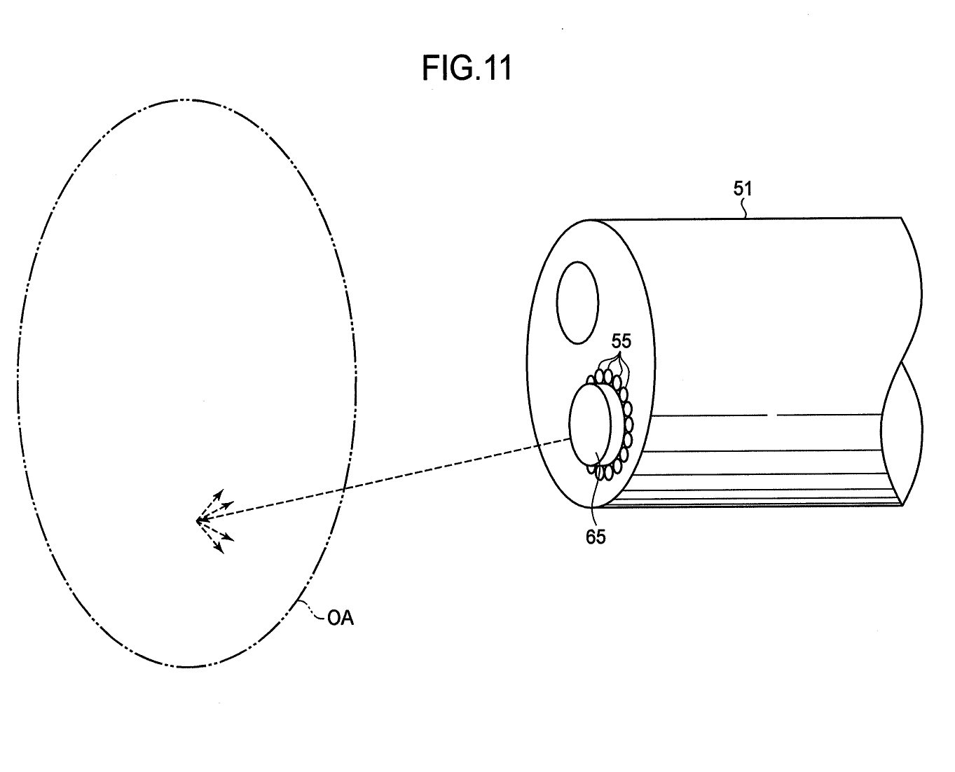

The white laser beam emitted from the illumination fiber 53 passes through the lens 65, and is shined on an individual point within the observation area (see “OA” in FIG. 11). The reflected light is scattered at that point. The scattered and reflected light is incident on the incident ends of the image fibers 55.

A plurality of the image fibers 55 are mounted in the scanning endoscope 50. The incident ends of the image fibers 55 are arranged around the lens 65 (see FIG. 11). The light that is scattered and reflected from the point in the observation area is incident on all the image fibers 55.

The reflected light incident on the incident ends of the image fibers 55 is transmitted to the emission ends of the image fibers 55. As described above, the emission ends of the image fibers 55 are optically connected to the light-capturing unit 21. The reflected light transmitted to the emission ends is incident on the light-capturing unit 21.

The light-capturing unit 21 detects the amounts of red, green, and blue light components in the reflected light, and generates pixel signals according to the amounts of the light components. The pixel signals are transmitted to the image-processing circuit 24.

The image-processing circuit 24 estimates the points where the white laser beam is shined on the basis of signals used to control the scanning driver 22. In addition, the image-processing circuit 24 stores the received pixel signals at the address of the image memory 27 that corresponds to the estimated points.

As described above, the observation area is scanned with the white laser beam, pixel signals are generated on the basis of the reflected light at the respective points illuminated with the white laser beam, and the generated pixel signals are stored at the addresses corresponding to the points. The image signal corresponding to the observation area comprises the pixel signals corresponding to the points from the scan-start point to the scan-end point. As described above, the image-processing circuit 24 carries out predetermined image processing on the image signal. After undergoing predetermined image processing, the image signal is transmitted to the monitor 11.

In the above first embodiment, the illumination fiber can be moved toward or away from the distal end of the insertion tube 51 along the first direction, which is the longitudinal direction of the illumination fiber 53. As described above, by moving the illumination fiber 53 along the first direction, a user can change the scanning speed and the scanned area.

By adjusting the scanning speed, different types of observation targets can be scanned more appropriately according to the type of the observation area. For example, if an observation target moves quickly, the motion resolution can be increased by capturing an image with a higher scanning speed. On the other hand, if a contour of an observation target is fine, a detailed image can be displayed by capturing an image with a lower scanning speed.

The braking period (see FIG. 6) can be reduced by changing the scanning speed during the braking period. By reducing the braking period, a frame rate can be increased to increase the motion resolution.

Next, a scanning endoscope of the second embodiment is explained. The primary difference between the second embodiment and the first embodiment is the structure of the fiber driving unit. The second embodiment is explained mainly with reference to the structures that differ from those of the first embodiment. Here, the same index numbers are used for the structures that correspond to those of the first embodiment.

The scanning endoscope 10 of the second embodiment comprises the illumination fiber 53, the fiber driving unit, the image fibers 55, and other components, as in the first embodiment. The fiber driving unit of the second embodiment is mounted at the distal end of the insertion tube 51, as in the first embodiment.

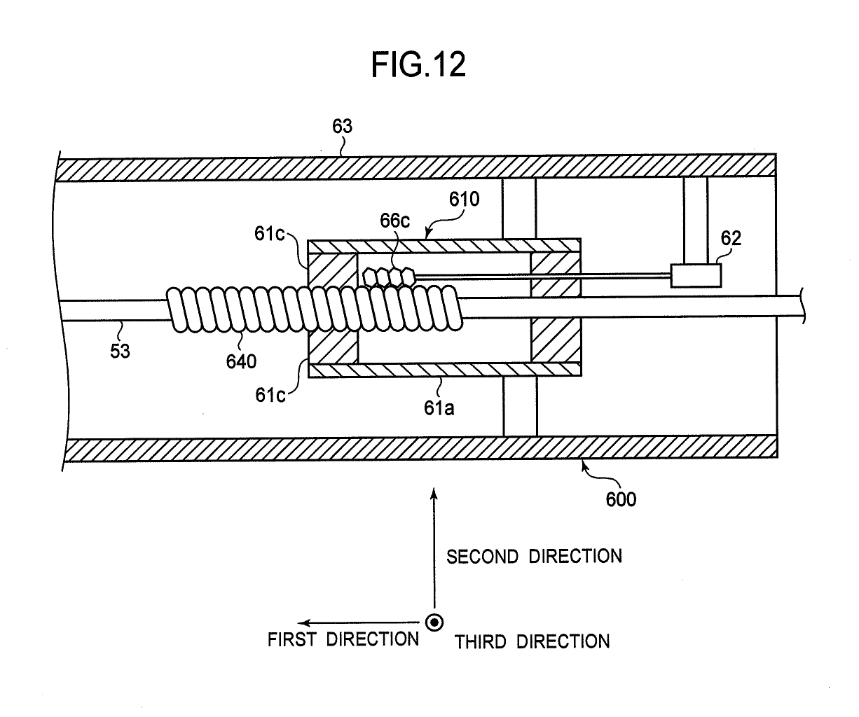

As shown in FIG. 12, the fiber driving unit 600 comprises the actuator 610, the motor 62, the rigid tube 63, the fiber supporter 640, and other components, as in the first embodiment. The structures of the hollow tube 61a, the piezoelectric elements 61b, and the rigid tube 63 are the same as those in the first embodiment.

The fiber supporter 640 is made of metal and formed as a coil spring so that the inside diameter of the coil is practically equal to the outside diameter of the illumination fiber 53, as in the first embodiment. The internal surface of the hollow tube 61a is threaded with an internal thread 61c at the end nearest to the distal end of the insertion tube 53, unlike in the first embodiment. The internal thread 61c is formed so that the internal thread 61c meshes with the outside surface of the coil spring of the fiber supporter 640. In addition, the internal thread 61c and the fiber supporter 640 are configured so that the axial direction of the coil of the fiber supporter 640 is parallel to the first direction.

The fiber supporter 640 is formed so that the fiber supporter 640 is longer than the internal thread 61c in the first direction. The fiber supporter 640 is screwed into and supported by the internal thread 61c so that the fiber supporter 640 protrudes from both ends of the internal thread 61c.

The illumination fiber 53 passes through the coil of the fiber supporter 640, as in the first embodiment. The illumination fiber 53 is fixed at the end of the hollow tube 61a that is furthest from the distal end of the insertion tube 51, unlike in the first embodiment.

The illumination fiber 53 is flexible, as in the first embodiment. The illumination fiber 53 bends toward the second and/or third directions when pushed by the actuator 610 via the fiber supporter 640. In addition, the observation area is scanned with light that is emitted from the moving emission end of the illumination fiber 53, which is moved by the pushing force exerted on it by the actuator 610, as in the first embodiment.

The motor 62 is fixed in the rigid tube 63 at a position that is further from the distal end of the insertion tube 51 than the actuator 610, as in the first embodiment. The motor 62 is connected to the third worm gear 66c. The motor 62 rotates the third worm gear 66c on a straight line parallel to the first direction.

The third worm gear 66c is configured so that the third worm gear 66c can mesh together with the coil spring of the fiber supporter 640. In addition, the third worm gear 66c and the motor 62 are arranged so that the third worm gear 66c makes direct contact with the exterior surface of the coil spring of the fiber supporter 640.

When the motor 62 rotates the third worm gear 66c, the fiber supporter 640 is rotated. By rotating, the fiber supporter 640 moves along the first direction toward or away from the distal end of the insertion tube 51.

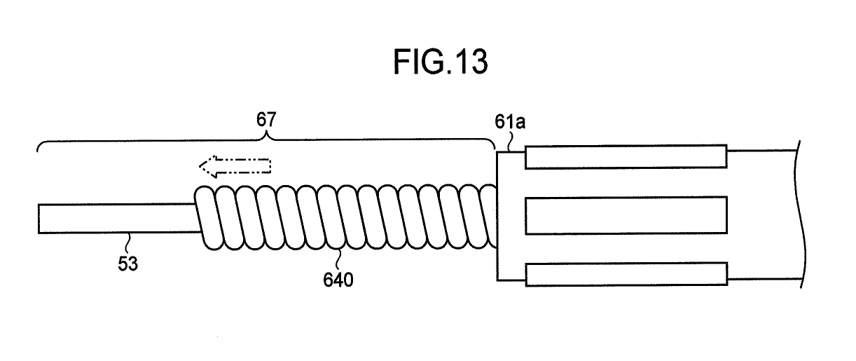

When the supporting block 640 is moved toward the distal end of the insertion tube 51 (see FIG. 13), the center of mass of the protruding section 67 shifts toward the distal end of the insertion tube 51. Accordingly, the resonant frequency of the protruding section 67 is decreased and the scanning speed is reduced.

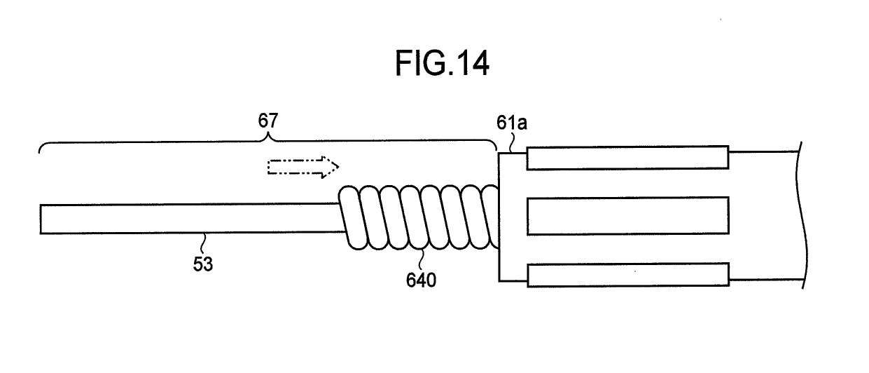

When the supporting block 640 is moved away from the distal end of the insertion tube 51 (see FIG. 14), the center of mass of the protruding section 67 shifts away from the distal end of the insertion tube 51. Accordingly, the resonant frequency of the protruding section 67 increases and the scanning speed also increases.

In the above second embodiment, the fiber supporter 640 can be moved along the first direction toward or away from the distal end of the insertion tube 51. The illumination fiber 53 is not moved in the first direction and the scanned portion of the observation area does not vary, unlike in the first embodiment. Accordingly, the second embodiment is preferable if adjusting only the scanning speed is required.

Next, a scanning endoscope of the third embodiment is explained. The primary difference between the third embodiment and the first embodiment is the structure of the fiber driving unit. The third embodiment is explained mainly with reference to the structures that differ from those of the first embodiment. Here, the same index numbers are used for the structures that correspond to those of the first embodiment.

The scanning endoscope 10 of the third embodiment comprises the illumination fiber 53, the fiber driving unit, the image fibers 55, and other components, as in the first embodiment. The fiber driving unit of the third embodiment is mounted at the distal end of the insertion tube 51, as in the first embodiment.

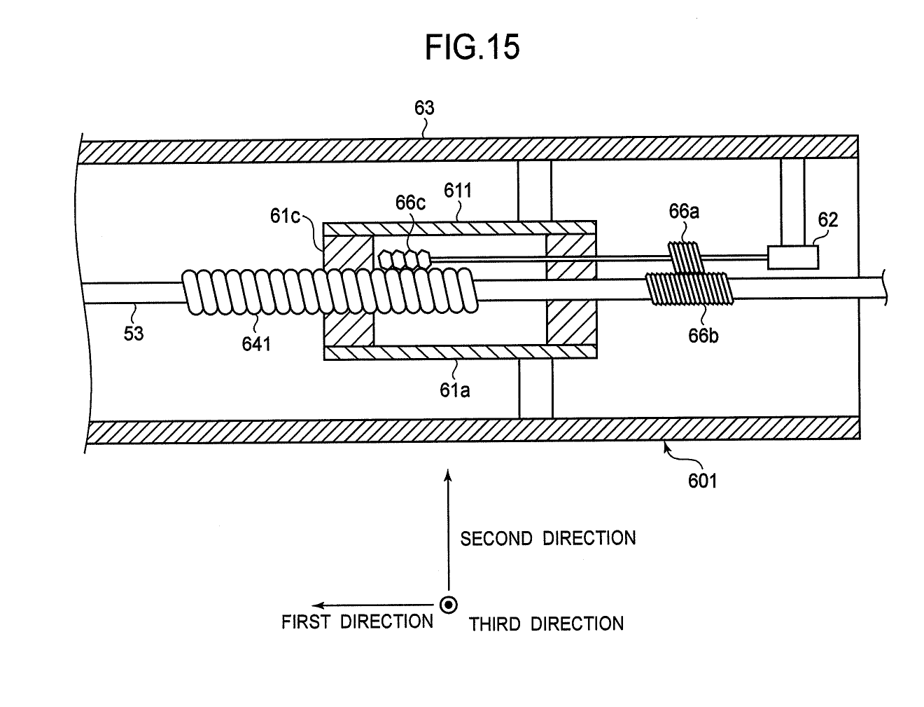

As shown in FIG. 15, the fiber driving unit 601 comprises the actuator 611, the motor 62, the rigid tube 63, the fiber supporter 641, and other components, as in the first embodiment. The structures of the hollow tube 61a, the piezoelectric elements 61b, and the rigid tube 63 are the same as those in the first embodiment.

The fiber supporter 641 is made of metal and configured as a coil spring with an inside diameter of the coil is practically equal to the outside diameter of the illumination fiber 53, as in the first embodiment. The internal surface of the hollow tube 61a is threaded with an internal thread 61c at the end of the hollow tube 61a nearest to the distal end of the insertion tube 53, as in the second embodiment. The internal thread 61c is formed so that the internal thread 61c meshes with the outside surface of the coil spring of the fiber supporter 641, as in the second embodiment. In addition, the internal thread 61c and the fiber supporter 641 are configured so that the axial direction of the coil of the fiber supporter 641 is parallel to the first direction.

The fiber supporter 641 is formed so that the fiber supporter 641 is longer than the internal thread 61c in the first direction, as in the second embodiment. The fiber supporter 641 is screwed into and supported by the internal thread 61c so that the fiber supporter 641 protrudes from both ends of the internal thread 61c.

The illumination fiber 53 passes through the coil of the fiber supporter 641, as in the first embodiment. The illumination fiber 53 is supported by the fiber supporter 641 as the emission end of the illumination fiber 53 protrudes from the fiber supporter 641, as in the first embodiment. In addition, the illumination fiber 53 is not fixed to the fiber supporter 641 and is free to move in the first direction, as in the first embodiment.

The illumination fiber 53 is flexible, as in the first embodiment. The illumination fiber 53 bends toward the second and/or third directions when pushed by the actuator 611 via the fiber supporter 641. In addition, the observation area is scanned with light that is emitted from the moving emission end of the illumination fiber 53, which is moved by the pushing force exerted upon it by the actuator 611, as in the first embodiment.

The motor 62 is fixed in the rigid tube 63 at a position that is further from the distal end of the insertion tube 51 than the actuator 611, as in the first embodiment. The motor 62 is connected to the first and third worm gears 66a and 66c, unlike in the first embodiment. The motor 62 rotates the first and third worm gears 66a and 66c on a straight line parallel to the first direction.

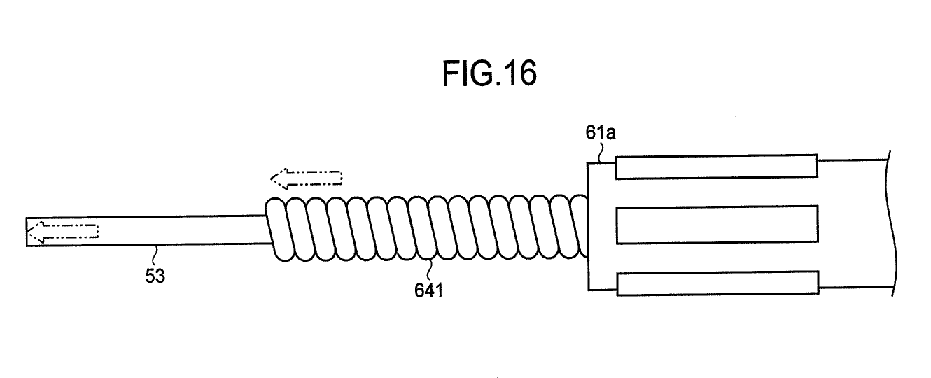

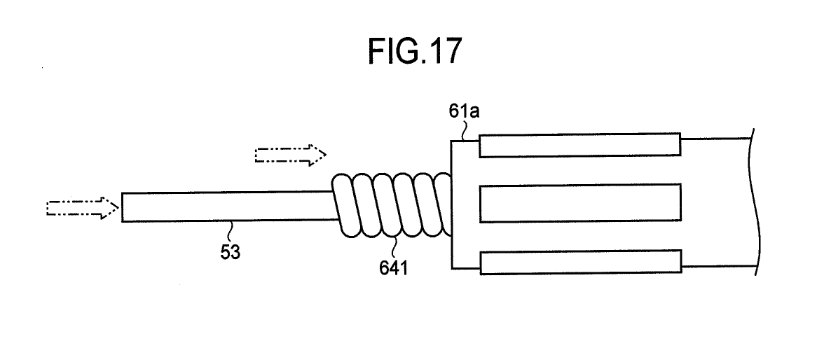

When the motor 62 rotates the first and third worm gears 66a and 66c, the second worm gear 66b and the fiber supporter 641 are rotated. By rotating, the second worm gear 66b, the fiber supporter 641, and the illumination fiber all move along the first direction toward (see FIG. 16) or away from (see FIG. 17) the distal end of the insertion tube 51.

By moving the illumination fiber 53 in either direction along the first direction, the scanning speed and the scanned area can be adjusted, as in the first embodiment. By moving the fiber supporter 641 in either direction along the first direction, the scanning speed can be adjusted, as in the second embodiment.

The rate of change in the scanning speed can be different from the rates of change in the first and second embodiments, because the illumination fiber 53 and the fiber supporter 641 are simultaneously moved toward or away from the distal end in the first direction, unlike in the first and second embodiments.

By changing the certain properties, such as the pitches of the first-third worm gears 66a, 66b, and 66c, and the fiber supporter 641, the direction and/or distance of movement of the illumination fiber 53 and/or the fiber supporter 641 per rotation of the motor 62 can be adjusted.

Next, a scanning endoscope of the fourth embodiment is explained. The primary difference between the fourth embodiment and the first embodiment is the structure of the fiber driving unit. The fourth embodiment is explained mainly with reference to the structures that differ from those of the first embodiment. Here, the same index numbers are used for the structures that correspond to those of the first embodiment.

The scanning endoscope 10 of the fourth embodiment comprises the illumination fiber 53, the fiber driving unit, the image fibers 55, and other components, as in the first embodiment. The fiber driving unit of the fourth embodiment is mounted at the distal end of the insertion tube 51, as in the first embodiment.

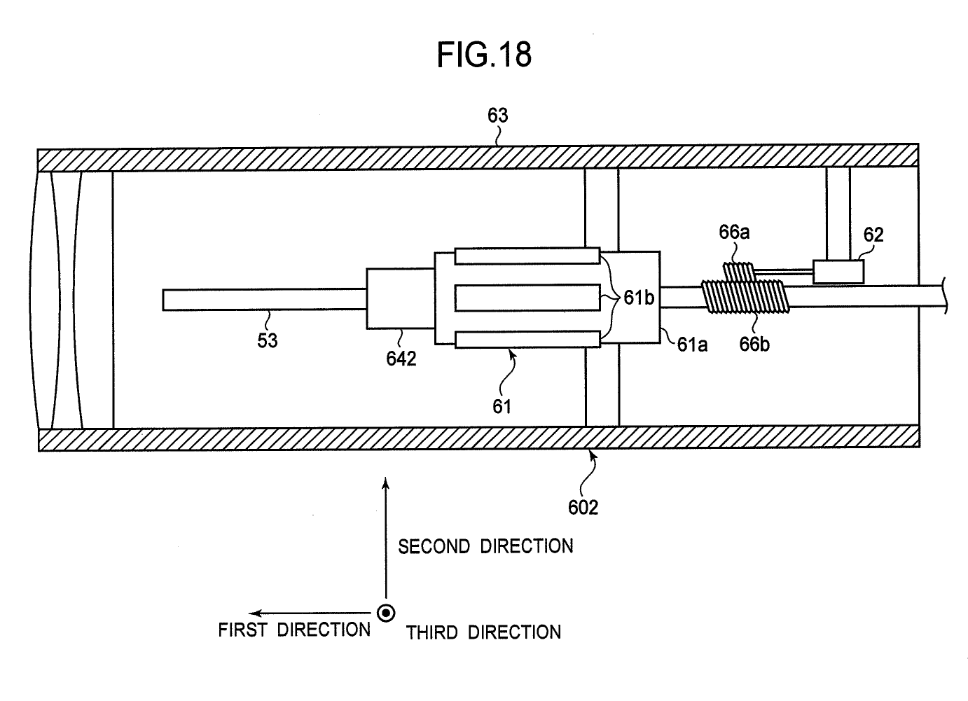

As shown in FIG. 18, the fiber driving unit 602 comprises the actuator 61, the motor 62, the rigid tube 63, the fiber supporter 642, and other components, as in the first embodiment. The structures of the actuator 61 and the rigid tube 63 are the same as those in the first embodiment.

The fiber supporter 642 is made of elastic material and formed as a tube so that the outside and inside diameters of the fiber supporter 642 are practically equal to the inside diameter of the hollow tube 61a and the outside diameter of the illumination fiber 53, respectively, unlike in the first embodiment. One end of the fiber supporter 642 is fixed inside the hollow tube 61a so that the axes of the fiber supporter 642 and the hollow tube 61a coincide with each other, and the other end of the fiber supporter 642 protrudes from the hollow tube 61a toward the distal end of the insertion tube 51.

The illumination fiber 53 passes through the fiber supporter 642. The illumination fiber 53 is supported by the fiber supporter 642 as the emission end of the illumination fiber 53 protrudes from the fiber supporter 642. The illumination fiber 53 is not fixed to the fiber supporter 642 and is free to move along the first direction, as in the first embodiment.

The illumination fiber 53 is flexible, as in the first embodiment. The illumination fiber 53 bends toward the second and/or third directions when pushed by the actuator 61 via the fiber supporter 642. In addition, the observation area is scanned with light that is emitted from the moving emission end of the illumination fiber 53, which is moved by the pushing force exerted on it by the actuator 61, as in the first embodiment.

The structures of the motor 62 and the first and second worm gears 66a and 66b are the same as those in the first embodiment. Accordingly, when the motor 62 rotates the first worm gear 66a, the second worm gear 66b is rotated and the illumination fiber 53 is moved in either direction along the first direction.

By moving the illumination fiber 53 along the first direction, the scanning speed and the scanned area can be adjusted, as in the first embodiment. Although the fiber supporter is a coil spring in the first-third embodiments, the fiber supporter is not limited to a coil spring. For example, if the fiber supporter is made of an elastic material as in the fourth embodiment, the same effect can be achieved in the first embodiment.

The motor 62 moves the illumination fiber 53 along the first direction via the first and second worm gears 66a and 66b in the first, third, and fourth embodiments. And the motor 62 moves the fiber supporter 640 and 641 along the first direction via the third worm gear 66c in the second and third embodiments. However, the illumination fiber 53 and/or the fiber supporter 640 and 641 may be moved along the first direction by using another mechanism. For example, the illumination fiber 53 and/or the fiber supporter 640 and 641 can be moved along the first direction by using a wire.

The actuator 61, 610, and 611 is deflected by expanding and contracting the piezoelectric elements 61b along the first direction, and a pushing force is exerted on the side of the illumination fiber 53 by the hollow tube 61b constituting the actuator 61, 610, and 611, in the first-fourth embodiments. However, any other kinds of actuators can be adopted as long as the actuator moves the illumination fiber 53 by pushing the side of the illumination fiber 53.

The structure that enables the illumination fiber and/or the fiber supporter to be moved along the longitudinal direction of the illumination fiber is adapted to a general scanning endoscope in the first-fourth embodiment. However, the structure can be adapted to any other kinds of scanning endoscopes.

For example, if the structure that enables the illumination fiber to be moved along the longitudinal direction of the illumination fiber is adapted to a confocal scanning endoscope, it would be able to adjust the focal depth. By capturing optical images at various focal depths and producing an image, a three-dimensional structure of a target to be observed could be displayed clearly.

The fiber supporter 64 is made of metal in the first embodiment. The fiber supporter 642 is made of elastic material in the fourth embodiment. However, the fiber supporter can be made of another material. Or, the illumination fiber 53 can be directly supported by the hollow tube 61a without the fiber supporter. Of course, damage to the illumination fiber can be reduced by transmitting the restoring force through the elastic deformation of the fiber supporter as in the first-fourth embodiments.

The fiber supporter 640 and 641 is made of metal in the second and third embodiments. However, the fiber supporter can be made of another material.

A singular motor 62 rotates the first and third worm gears 66a and 66c in the third embodiment. However, different motors can rotate the first and third worm gears 66a and 66c separately. Although the structure is complex, the illumination fiber 53 and the fiber supporter 641 can be moved separately.

The illumination fiber 53 is moved so that the emission end of the illumination fiber 53 traces a predetermined spiral course in the above first-fourth embodiments. However, a course to be traced is not limited to a spiral course. The illumination fiber 53 can be moved so that the emission end traces another predetermined course.

Although the embodiments of the present invention have been described herein with reference to the accompanying drawings, obviously many modifications and changes may be made by those skilled in this art without departing from the scope of the invention.

The present disclosure relates to subject matter contained in Japanese Patent Application No. 2009-005163 (filed on Jan. 13, 2009), which is expressly incorporated herein, by reference, in its entirety.

1. A scanning endoscope comprising:
a light transmitter that transmits light received at a first incident end to a first emission end, the light transmitter emitting a beam of the light exiting the first emission end, the light transmitter being flexible, a longitudinal direction of the light transmitter being a first direction;
a first actuator that is mounted near the first emission end, the first actuator bending the light transmitter in a second direction by pushing a side of the light transmitter toward the second direction, the second direction being perpendicular to the first direction;
a weight that is movable from the first actuator to the first emission end along the first direction, the weight changing a position of the center of mass of a protruding section by moving in unison with the light transmitter in the second direction when the first actuator bends the light transmitter, the protruding section being a section of the illumination fiber that is extended from the first actuator; and
a second actuator that moves the weight in either direction along the first direction.

2. A scanning endoscope according to claim 1, wherein the weight has a length in the first direction, the weight is made of an elastic material, the weight is positioned between the light transmitter and the first actuator, and the weight transmits a pushing force from the first actuator to a side of the light transmitter as the weight is deformed elastically in the second direction.

3. A scanning endoscope according to claim 2, further comprising a worm gear that meshes with the weight that is shaped as a coil spring, the weight being moved in either direction along the first direction by the second actuator rotating the worm gear.

4. A scanning endoscope according to claim 1, further comprising a third actuator that moves the light transmitter in either direction along the first direction.

5. A scanning endoscope according to claim 1, wherein the second actuator moves the light transmitter in either direction along the first direction.

6. A scanning endoscope according to claim 5, wherein the second actuator moves the weight and the light transmitter different distances.

7. A scanning endoscope comprising:
a light transmitter that transmits light received at a first incident end to a first emission end, the light transmitter emitting a beam of the light exiting the first emission end, the light transmitter being flexible, a longitudinal direction of the light transmitter being a first direction;
a first actuator that is mounted near the first emission end, the first actuator bending the light transmitter in a second direction by pushing a side of the light transmitter toward the second direction, the second direction being perpendicular to the first direction; and
a second actuator that moves the light transmitter in either direction along the first direction.
